(19) United States
(12) Patent Application Publication
(10) Pub. No.: US 2022/0195509 A1
(43) Pub. Date: Jun. 23, 2022

(54) Systems and Methods for Ratiometric and Multiplexed Isothermal Amplification of Nucleic Acids

(71) Applicant: The Board of Trustees of the Leland Stanford Junior University, Stanford, CA (US)

(72) Inventors: Rhiju Das, Stanford, CA (US); Kevin Shih, Stanford, CA (US); Matthew Adrianowycz, Stanford, CA (US)

(73) Assignee: The Board of Trustees of the Leland Stanford Junior University, Stanford, CA (US)

(21) Appl. No.: 17/594,868

(22) PCT Filed: May 1, 2020
(86) PCT No.: PCT/US20/31011

§ 371 Date: Nov. 1, 2021

Related U.S. Application Data
(60) Provisional application No. 62/841,689, filed on May 1, 2019.

(51) Int. Cl. C12Q 1/6851; C12Q 1/6883

(52) U.S. Cl. CPC C12Q 1/6851; C12Q 1/6883

(57) ABSTRACT

Systems and methods for isothermal amplification of nucleic acids in a ratiometric manner are disclosed. Methods of isothermal amplification of nucleic acids are convenient for areas that lack reliable electricity and/or funding for precision lab equipment. However, these methods typically result in non-ratiometric amplification of target sequences, thus preventing quantitative analysis or application of the results, such as for diagnostic testing. Systems and methods herein provide a method of isothermal amplification that ratiometrically amplifies target sequences, thus expanding the reach of diagnostics into remote and/or economically disadvantaged areas.

### CROSS REFERENCE TO RELATED APPLICATIONS

This application claims priority to U.S. Provisional Application Ser. No. 62/841,689, entitled “Systems and Methods for Ratiometric and Multiplexed Isothermal Amplification of Nucleic Acids” to Das et al., filed May 1, 2019; the disclosure of which is herein incorporated by reference in its entirety.

### FIELD OF THE DISCLOSURE

The present disclosure relates to nucleic acid amplification, and in particular methods and systems for amplifying nucleic acids in a ratiometric, multiplexed, and isothermal manner.

### BACKGROUND OF THE DISCLOSURE

There are several diseases, such as tuberculosis, sepsis, and HIV, which remain deadly worldwide and disproportionately affect developing countries. Often, treatments for these diseases are available and the risk of death can be greatly mitigated if patients are identified early in disease progression. Organizations such as the World Health Organization have identified cheap point-of-care diagnostics as critical tools needed for combatting these global health threats. (See P. K. Drain, et al., Diagnostic point-of-care tests in resource-limited settings. Lancet Infect Dis. 14, 239-249 (2014); the disclosure of which is herein incorporated by reference in its entirety.)

Many of these diseases can now be diagnosed using the ratios of ribonucleic acids (RNAs) extracted from patient blood, saliva, urine, or other bodily fluids. The main method used to determine gene ratios in patient samples is quantitative polymerase chain reaction (qPCR), which monitors the amplification of target genes through several cycles of PCR to determine either the relative or absolute levels of genes in the sample. However, qPCR relies on costly reagents and specialized equipment (e.g., thermal cyclers) that can be prohibitively expensive, difficult to transport, and require a reliable supply of electricity to function, making it unsuitable as a point-of-care diagnostic strategy in areas where these diseases are very problematic.

While several amplification techniques exist, such as strand displacement amplification (SDA), recombinase polymerase amplification (RPA), loop-mediated isothermal amplification (LAMP), and nucleic acid sequence based amplification (NASBA), which are carried out isothermally (e.g., at constant temperatures) and do not require specialized equipment, such as thermal cyclers, these methods do not preserve gene ratios during amplification, thus making them unsuitable for many diagnostic purposes.

### SUMMARY OF THE DISCLOSURE

This summary is meant to provide examples and is not intended to be limiting of the scope of the invention in any way. For example, any feature included in an example of this summary is not required by the claims, unless the claims explicitly recite the feature. Also, the features described can be combined in a variety of ways. Various features and steps as described elsewhere in this disclosure can be included in the examples summarized here.

In one embodiment, a method for ratiometric amplification of specific nucleic acids includes obtaining or having obtained a sample, wherein the sample contains nucleic acids, and performing an amplification reaction on the sample to amplify one or more target sequences in the sample by combining the sample with reaction components, wherein the reaction components comprise at least one target specific primer, nucleotides, and at least one polymerase, and incubating the sample and the reaction components.

In another embodiment, the incubating step is performed isothermally.

In a further embodiment, the incubating step is performed with an initial heating step, then incubated isothermally.

In yet another embodiment, the method further includes analyzing an output of the amplification reaction.

In a still further embodiment, the method further includes analyzing an output of the amplification reaction to identify one or more target sequences.

In still another embodiment, the analyzing step is performed using electrophoresis.

In a yet further embodiment, the electrophoresis is selected from the group consisting of gel electrophoresis and capillary electrophoresis.

In a further embodiment again, the electrophoresis is capable of quantitatively analyzing the output components.

In another embodiment again, the amplification reaction is selected from the group consisting of: nucleic acid sequence based amplification (NASBA), Loop-mediated isothermal amplification, Recombinase Polymerase Amplification, Rolling Circle Amplification, and Nicking Enzyme Amplification Reaction.

In a further additional embodiment, the analyzing step includes diagnosing an individual for a disease or disease state.

In another additional embodiment, the disease or disease state is selected from the group consisting of HIV, tuberculosis, and sepsis.

In a still yet further embodiment, the reaction components include dNTPs, NTPs, a buffer, at least one primer, and a transcriptase.

In still yet another embodiment, the components further include a reverse transcriptase.

In a still further embodiment again, the transcriptase is an RNA polymerase.

In still another embodiment again, the components further include a nuclease.

In a still further additional embodiment, the nuclease is RNase H.

In still another additional embodiment, the dNTPs are a limiting reagent in the reaction to maintain ratiometric amplification of the template nucleic acids in the sample.

In a yet further embodiment again, the dNTP concentration is no greater than ⅕× concentration for a standard amplification reaction.

In a yet further additional embodiment, the biological sample is selected from the group consisting of saliva, urine, and blood.

### BRIEF DESCRIPTION OF THE DRAWINGS

FIG. 1 illustrates a method of nucleic acid sequence based amplification (NASBA) in accordance with various embodiments.

FIG. 2 illustrates a method of performing or using a NASBA reaction or kit in accordance with various embodiments.

FIGS. 3A-3B illustrate kinetiscope output of computational testing of several NASBA reactions under different template concentrations in accordance with various embodiments.

FIGS. 4A-4B illustrate kinetiscope output of computational testing of several NASBA reactions under different NTP concentrations in accordance with various embodiments.

FIGS. 5A-5B illustrate kinetiscope output of computational testing of several NASBA reactions under different primer concentrations in accordance with various embodiments.

FIGS. 6A-6B illustrate kinetiscope output of computational testing of several NASBA reactions under different dNTP concentrations in accordance with various embodiments.

FIGS. 7A-7F illustrate kinetiscope output of computational testing of several NASBA reactions under different dNTP concentrations in accordance with various embodiments.

FIG. 8 illustrates kinetiscope output of computational testing of a NASBA reactions under various reaction conditions in accordance with various embodiments.

FIGS. 9A-9B illustrate parameters used in kinetiscope computations of several NASBA reactions in accordance with various embodiments.

FIG. 10 illustrates kinetiscope version used in computational testing of several NASBA reactions in accordance with various embodiments.

### DETAILED DESCRIPTION OF THE DISCLOSURE

Generally, traditional NASBA reactions utilize gene specific primers, reverse transcriptase, RNase H, and T7 RNA polymerase to exponentially amplify the antisense versions of target input RNAs. (See J. Compton, Nucleic acid sequence-based amplification. Nature. 350, 91-92 (1991); the disclosure of which is herein incorporated by reference in its entirety.) However, a previous study of NASBA kinetics used electrochemiluminescence-based quantification and computer modelling, which suggest that RNA amplification occurs in two phases, and the primers were found to be the limiting reagent that dictates when each phase occurs. (See J. J. A. M. Weusten, et al., Principles of quantitation of viral loads using nucleic acid sequence-based amplification in combination with homogeneous detection using molecular beacons. Nucleic Acids Res. 30(6), e26 (2002); the disclosure of which is herein incorporated by reference in its entirety.)

In the first phase, antisense RNA is exponentially formed but quantitatively relevant amounts do not accumulate due to RNase H destroying RNA in RNA-DNA duplexes. When the primers are depleted, the reaction proceeds to the second phase. Now, the cDNA levels are at a maximum, RNase H is no longer degrading RNA, and antisense RNA is produced at a linear rate proportional the amount of cDNA. Because primers are the limiting reagent, in a multiplexed reaction the amplification of separate target genes will reach this linear amplification phase at different time points during the reaction based on initial template concentrations. However, primer concentrations determine maximum cDNA levels and thus the rates of linear amplification for all genes will be roughly the same. In this scenario, where linear amplification of different genes begin at different times but all have the same rate, the initial ratios of the input genes will not be conserved.

Turning now to the drawings and data, embodiments of the invention are generally directed to methods of nucleic acid amplification and applications thereof. Many embodiments are directed to methods of multiplexed NASBA where the initial ratios of target genes are quantitatively maintained post amplification. In certain embodiments, the ratiometric amplification will be achieved by ensuring that dNTPs, instead of primers, are the limiting reagent. By limiting dNTPs in many embodiments, cDNA generation of target sequences is limited and therefore “linked” by a shared dNTP pool. With the methodology of these embodiments, the first phase of the reaction still undergoes exponential antisense RNA production and cDNA generation. When dNTPs are depleted, cDNA generation for all target genes stops, RNase H will stop degrading RNA, and unlike in classical NASBA the phase two linear amplification of different genes begins at the same time. Each target's phase one amplification is given the same amount of time, so the relative cDNA levels of each gene should be proportional to the initial ratios of the input RNAs. The linear amplification in phase two is proportional to cDNA level, conserving this difference until the reaction is stopped and ensuring that input RNA ratios are maintained post amplification.

Additional embodiments are used as diagnostic tools to detect specific ratios of genes or gene products (e.g., RNAs) that exist in a sample. Additionally, the isothermal amplification allows for some embodiments to be used in remote areas without using sophisticated, sensitive, and/or expensive equipment, such as thermal cyclers and/or sequencers. Because of the isothermal nature, many embodiments allow for the use of more common and/or less expensive equipment, including water baths or even by using body heat to allow the amplification reaction to proceed.

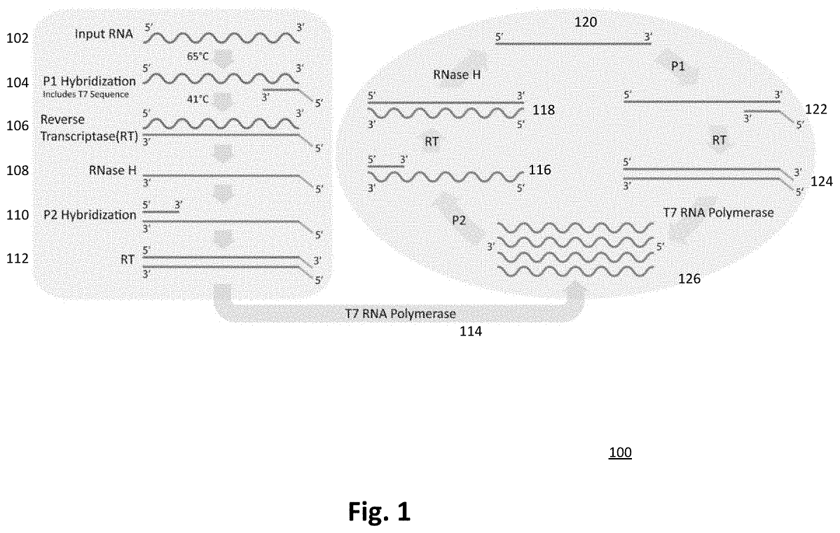

Turning to FIG. 1, a method of a nucleic acid sequence based amplification (NASBA) 100 is illustrated in accordance with some embodiments. In method 100, input nucleic acids are obtained at step 102 in certain embodiments. In certain embodiments, the input is RNA, while in other embodiments, the input is deoxyribonucleic acid (DNA). In various embodiments, the input has been isolated through known methods including commercial kits, while some embodiments will involve a whole sample, such as blood, urine, saliva, or other fluid or tissue sample. In some embodiments, the sample will be heated to reduce or eliminate any secondary structures or nucleic acid pairing formed in the input sample.

At step 104, a first primer is hybridized to the input nucleic acid in various embodiments. In some embodiments, this step is accomplished by adding the first primer to the input nucleic acid and allowing a region on the first primer to pair with its complementary region in the input nucleic acid. In some embodiments, the primer hybridization 104 is accomplished by heating the sample at or near (e.g., ±2° C.) the melting temperature (Tₘ) of the first primer. In certain embodiments, the heating to the melting temperature will be accomplished concurrently with or subsequently to any heating performed in step 102, such as if the input nucleic acid is heated to a temperature to reduce or eliminate secondary structures, the primer hybridization can be accomplished as the sample cools.

In many embodiments, the first primer includes a tag sequence. This tag sequence can include a common sequence among numerous amplicons for subsequent rounds of amplification, a functional sequence (e.g., transcription start site), or a unique sequence to identify a specific amplicon. In many embodiments, the tag sequence is a T7 promoter to allow a T7 RNA polymerase to transcribe a sequence into RNA.

At step 106, certain embodiments will synthesize a first DNA strand from the first primer hybridized in step 104. If using RNA as an input sample, the first strand is synthesized using a RNA-dependent DNA-polymerase, such as a reverse transcriptase, while embodiments using a DNA input sample will utilize a DNA-dependent DNA-polymerase. In many embodiments, the first strand will be “antisense” to any input strand, as it is the reverse complement to the input strand.

At step 108, the sample strand is removed from the first strand synthesized in step 106 in several embodiments. For embodiments utilizing RNA input samples, the input sample can be removed via enzymatic digestion, such as a ribonuclease, while embodiments utilizing DNA input samples can denature the strands via heat or another method known in the art. For RNA input samples, many embodiments will utilize RNase H, which hydrolyze RNA form DNA-RNA hybrids but will not hydrolyze single stranded RNA from the reaction.

At step 110, a second primer is hybridized to the first strand in many embodiments. The hybridization of the second primer will be complementary to a portion of the first strand. In many embodiments, the Tₘ of the second primer is at or near (e.g., ±2° C.) of the reaction temperature of the entire process to allow for annealing of the second primer.

Various embodiments will synthesize a second strand at step 112. In this process, a new DNA strand is synthesized using the synthesized first strand (from step 106) as a template, and creates a double stranded DNA molecule that possess the sequence of the input sample in addition to any tag sequences that were included on the first primer. In many embodiments, the first strand will be “sense” to the input strand, as it is transcribed from the reverse complement to the input strand. This polymerization will utilize a DNA polymerase that can be DNA-dependent or template ambivalent. In many embodiments, a reverse transcriptase is utilized to synthesize the second strand.

At step 114 of certain embodiments, antisense RNA strands are generated. In many embodiments, the antisense RNA strands will be generated using T7 RNA polymerase, which will utilize a transcription start site (e.g., a T7 promoter) that was added as a tag sequence as part of the first primer.

At step 116, numerous embodiments will hybridize the second primer to the antisense RNA strands generated in step 114, and a sense DNA strand will be synthesized off of the second primer, similar to the processes in steps 110 and 112.

At step 120 of many embodiments, the antisense RNA strands are removed from the newly synthesized sense DNA strands. This process can be accomplished in ways similar to the process of step 108, including the use of RNase H to remove RNA from a DNA-RNA hybrid molecule.

In several embodiments, the first primer is hybridized to the newly synthesized sense DNA strands at step 122, which is accomplished in means similar to those in step 104. Further, in many embodiments will synthesize a new antisense DNA strand at step 124, which will utilize the first primer and a polymerase, such as those used in step 110. At step 126 of numerous embodiments, additional antisense RNA strands will be generated, which can be accomplished in a similar manner as those utilized in step 114.

Additional embodiments are directed to kits for performing methods as describe above, including kits containing enzymes, such as transcriptases, reverse transcriptases, and nucleases, including T7 RNA polymerase, reverse transcriptase, and RNase H, as well as buffers, nucleoside triphosphates (NTPs), and deoxy nucleoside triphosphates (dNTPs).

Additionally, some kit embodiments will be customized for specific assays (e.g., a kit specific for diagnosing a disease, such as tuberculosis, sepsis, and/or HIV). Customized kits of various embodiments will also include primers for specific targets (e.g., genes or RNAs).

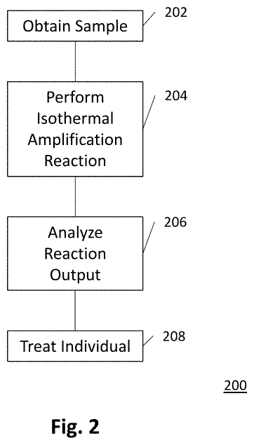

Further embodiments are directed to using or performing the methods or using kits, such as those described above. FIG. 2 illustrates a method 200 to treat an individual for a disease identified using a ratiometric isothermal amplification reaction. At 202, a sample is obtained in many embodiments. In many embodiments, the sample includes nucleic acids (e.g., RNA and/or DNA). In some embodiments, the sample is obtained from a biological source, including bodily fluids or tissues, such as saliva, blood, urine, fecal, epidermal, follicle, and/or any other applicable biological source from an individual. Additionally, environmental sources will be used in various embodiments to analyze environmental factors, and environmental sources can utilize water, soil, or aerial sources, which can contain trace amounts of various targets for use in quantifying environmental contaminants (e.g., bacteria, viruses, fungus, etc.). Nucleic acids will be isolated from the sample in various embodiments, such that the underlying DNA or RNA is isolated from cellular or environmental contamination. However, many embodiments will begin on the source as obtained, rather than having to isolate nucleic acids from the sample.

At 204 of many embodiments, an amplification reaction is performed on the biological sample to amply one or more target sequences. Some embodiments perform an isothermal amplification reaction. Various embodiments perform a NASBA reaction, such as illustrated in FIG. 1, while additional embodiments perform another isothermal amplification method, including the methods of Loop-mediated isothermal amplification, Recombinase Polymerase Amplification, Rolling Circle Amplification, Nicking Enzyme Amplification Reaction, and any other isothermal reaction known or developed.

In a number of embodiments, the sample (whether or not the nucleic acids are isolated from the sample) is combined with reaction components for the amplification reaction. Many embodiments will include target specific primers (forward and/or reverse primers), enzymes, nucleotides (e.g., NTPs and/or dNTPs), and buffer as relevant to a specific amplification reaction. In some embodiments, the primers further include adapter sequences. In a number of embodiments, the adapter sequences on the primers include transcription start sites for a particular enzyme, while some adapters are common primers used for subsequent PCR reactions. Enzymes included in many reactions are selected from DNA and/or RNA polymerases (e.g., Transcriptase, Pol I, Pol II, Pol II, Taq polymerase, T7 Polymerase, RNA Pol I, RNA Pol II, RNA Pol III, etc.), nucleases (e.g., RNase H, RNase A, etc.), and/or other modifying enzymes (e.g., helicases, ligases, etc.) which are relevant for the particular amplification reaction and/or to improve reaction results. Further embodiments include other nucleic acid molecules or chemicals to prevent amplification of short, “parasite” nucleic acids in the sample. Some embodiments include additional chemicals to assist in amplification to assist with stabilizing nucleic acids or reaction amplification, wherein these chemicals include DMSO, Magnesium (e.g., Mg²⁺), and/or any other chemical known to improve an amplification reaction.

In many embodiments, dNTPs are a limiting reagent in the reaction to maintain ratiometric amplification of the template nucleic acids in the sample. As such, numerous embodiments possess a dNTP concentration of ⅕×, 1/10×, 1/15×, 1/20×, 1/25×, 1/30×, 1/35×, 1/40×, 1/45×, 1/50×, 1/55×, 1/60×, 1/65×, 1/70×, 1/75×, 1/80×, 1/85×, 1/90×, 1/95×, 1/100× or lower relative to the amount used in a standard amplification reaction of the type performed. An amount used in a standard amplification reaction is generally recognized as the amount provided for in a published reaction or kit. For example, if a reaction requires a concentration of approximately 200 μM of dNTPs, certain embodiments possess a dNTP concentration of approximately 40 μM, approximately 20 μM, approximately 13.3 μM, approximately 10 μM, approximately 8 μM, approximately 6.7 μM, approximately 6.7 μM, approximately 5.7 μM, approximately 5 μM, approximately 4.4 μM, approximately 4 μM, approximately 3.6 μM, approximately 3.3 μM, approximately 3.1 μM, approximately 2.9 μM, approximately 2.7 μM, approximately 2.5 μM, approximately 2.4 μM, approximately 2.2 μM, approximately 2.1 μM, approximately 2 μM, or a lower concentration.

Once combined, many embodiments will incubate the sample to allow the underlying reaction to complete. In some embodiments, several embodiments will incubate the reaction at a single temperature (e.g., isothermally). Further embodiments will further include one or more initial heating steps, where the reaction is incubated at higher temperatures, such as described above regarding steps 102 and 104 of FIG. 1. Reaction temperatures will be dependent upon the specific reaction being performed and the enzymes, components, and/or reagents used in the reaction.

At 206 of many embodiments, the reaction outputs (e.g., target sequence amplicons) are analyzed. Analysis at 206 can include various forms, including identification of the individual outputs, such as confirmatory PCR, nucleic acid sequencing, fluorescence probing, and/or electrophoresis (e.g., gel electrophoresis and/or capillary electrophoresis). Additional embodiments quantitatively analyze the outputs to measure the ratios of the outputs by such methods as quantitative electrophoresis, fluorescence during and/or after the reaction, including the use of a plate reader or a thermal cycler outfitted for quantitative analysis (e.g., qPCR thermal cycler). Certain embodiments use denaturing gels (e.g., agarose or polyacrylamide gels) for quantitative electrophoresis. One or more fluorescence dyes or markers are used in many embodiments to visualize and/or quantify the outputs, including ROX, SYBR green, DAPI, Ethidium Bromide, and/or any other nucleic acid specific dye to identify the outputs.

In further embodiments, the analysis at 206 includes diagnosing a disease and/or disease state of the sample, such as identification of HIV, sepsis, and/or tuberculosis. In certain embodiments, the analysis is based on the ratios of specific targets identified in the sample.

In diagnostic uses, 208 of many embodiments will treat an individual from whom a sample is taken for a disease or disease state. Treatments can include treatment specific for the identified disease, such as antiviral drugs (including antiretroviral drugs), antibiotics, antifungals, and/or any other treatment that is appropriate for the identified disease or disease state.

It should be noted that several features of method 200 may be combined, omitted completed multiple times, and/or performed in a different order, as applicable for a particular use. For example, multiple samples may be obtained from a single individual and/or multiple samples from multiple sources may be combined for a single reaction pool. Additionally, multiple analysis or reaction steps may be performed, depending on necessity for either confirmation and/or additional output amplicons. Further, some embodiments combine analysis 206 with performing the amplification reaction 204 by the use of a quantitative thermocycler (e.g., qPCR machine), where simultaneous incubation and monitoring are performed.

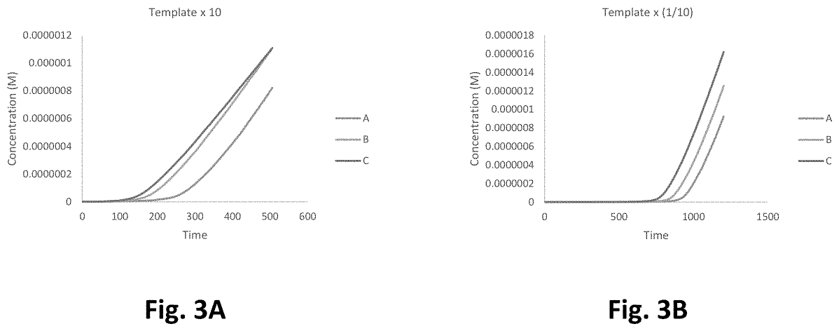

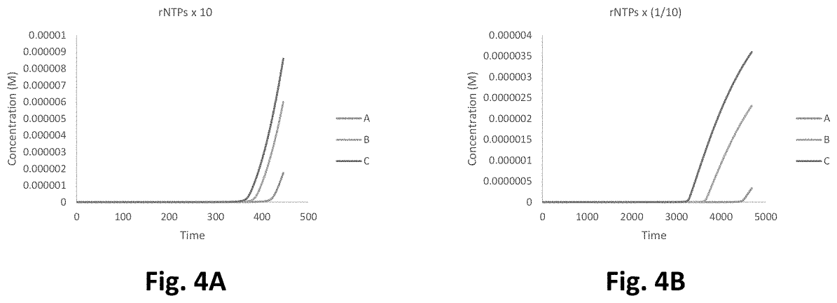

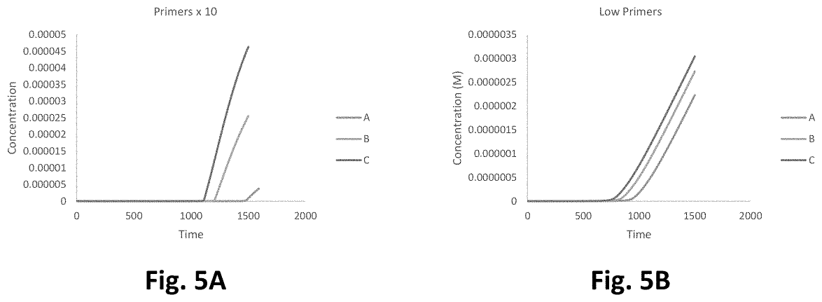

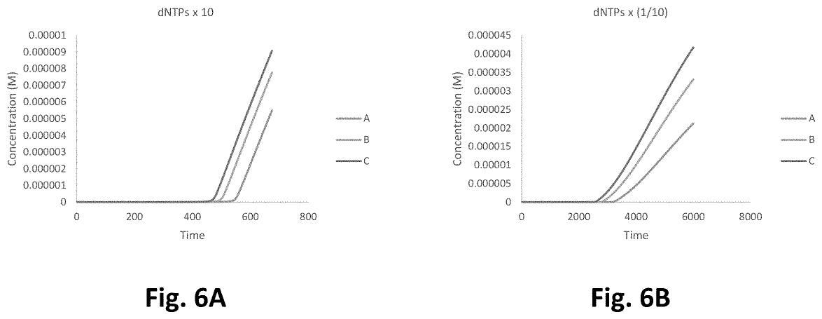

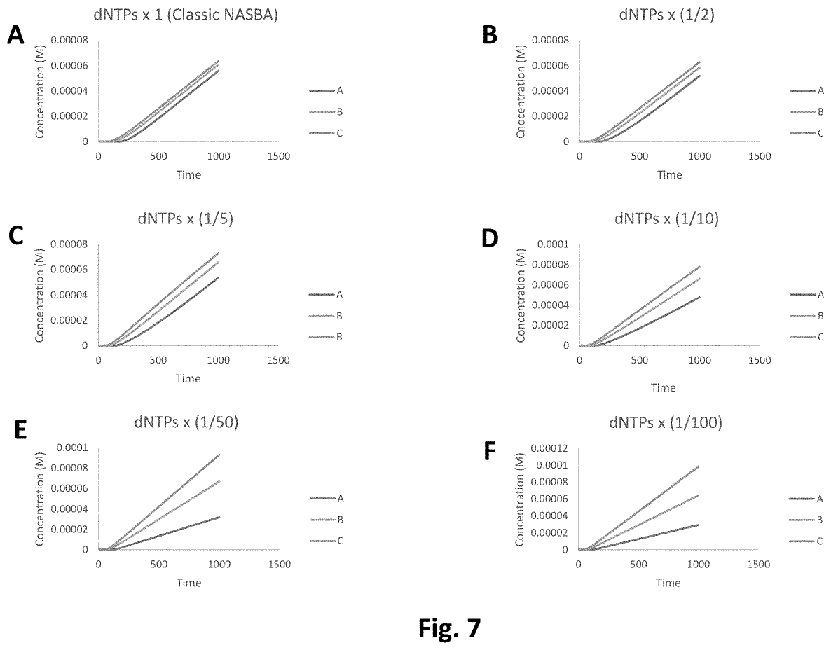

Turning now to FIGS. 3A-7F, computational output from varying reaction components relative to classic NASBA reactions are illustrated. In these embodiments, three templates (A, B, and C) are at 1× (template A), 2× (template B), and 3× (template C) relative concentrations. In FIGS. 3A-5B, the template concentration (FIGS. 3A and 3B), NTP concentration (FIGS. 4A and 4B), or primer concentration (FIGS. 5A and 5B) are varied in accordance with various embodiments but produce non-linear amplification of single stranded RNA (ssRNA) and do not match the relative concentration ratios of the input templates. However, numerous embodiments alter dNTP concentration, as shown in FIGS. 6A-7F, which does produce linear and ratiometric amplification of the starting templates as the dNTP concentration is reduced.

### Exemplary Embodiments

Although the following embodiments provide details on certain embodiments of the inventions, it should be understood that these are only exemplary in nature, and are not intended to limit the scope of the invention.

### Example 1: Computer Simulation of NASBA Kinetics

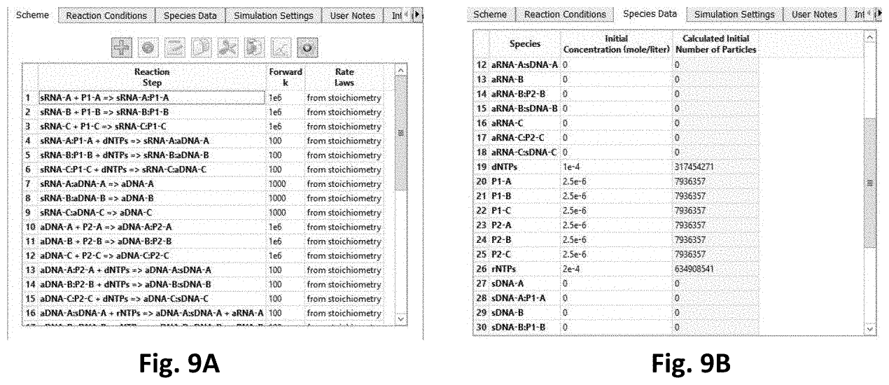

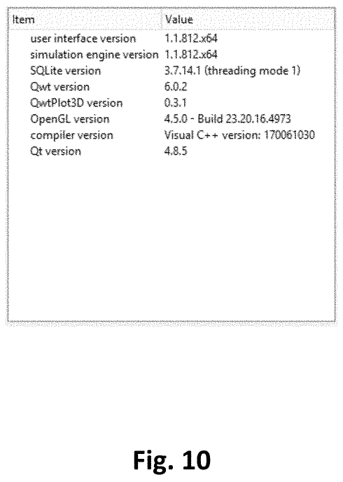

Methods: To model the kinetic properties of the system described and demonstrate that it may display the desired ratiometric amplification, a series of rules describing transitions between the possible species were postulated. From these transitions, a system of coupled, nonlinear differential equations could be written down, describing the change in concentration of each species with respect to time, then integrated numerically to study the behavior of the system over relevant timescale. The rules, including rate constants and initial concentrations are illustrated in FIGS. 9A and 9B, and the version of kinetiscope used in these calculations are show in FIG. 10.

Numerical integration was carried out by kinetic Monte Carlo (kMC), which only requires that the possible transitions and their relative rates be known. Such a scheme is comparable to usual numerical integration techniques for simple kinetic systems and significantly faster for stiff schemes. Our simulation was implemented in Kinetiscope, a freely available stochastic kinetic simulation program. (See W. D. Hinsberg, F. A Houle., Kinetiscope. www.hinsberg.net/kinetiscope (2015); the disclosure of which is herein incorporated by reference in its entirety.) Briefly, each step in the algorithm comprises generating two random numbers. The first determines the size of a time step and is drawn from an exponential probability distribution with mean of the inverse of the total reaction rate. The second determines the reaction that takes place in the time step with probability proportional to the relative rate of reaction. The simulation is then carried out by iterating the step described above until a defined stopping time is reached or the reactants decay to zero.

Results: FIGS. 3A-7F illustrate reaction simulations in kinetiscope producing graphical representations of single stranded RNA (ssRNA) concentration when the concentrations of various components (e.g., template, NTPs, primers, and dNTPs) are altered. In particular, FIGS. 3A and 3B illustrate changes in template concentration of 10× (FIG. 3A) and 1/10× (FIG. 3B) which maintain non-linear amplification at both concentration levels. Similarly, FIGS. 4A and 4B illustrate changes in NTP concentration of 10× (FIG. 4A) and 1/10× (FIG. 4B) which maintain non-linear amplification at both concentration levels. Also, FIGS. 5A and 5B illustrate primer concentration changes of 10× (FIG. 5A) and 1/10× (FIG. 5B) which also maintain non-linear amplification at both concentration levels.

However, FIGS. 6A and 6B illustrate 10× (FIG. 6A) and 1/10× (FIG. 6B) concentration of dNTPs, where the 1/10× concentration illustrated in FIG. 6B begins to show linear amplification of ssRNA of the three templates. Digging deeper, FIGS. 7A-7F illustrate even more changes of dNTP concentration of 1× (FIG. 7A), ½× (FIG. 7B), ⅕× (FIG. 7C), 1/10× (FIG. 7D), 1/50× (FIG. 7E), and 1/100× (FIG. 7F). As seen in these figures, when reaching lower concentrations of dNTPs (e.g., 1/50× and 1/100× (FIGS. 7E and 7F)), the amplification of ssRNA becomes linear and ratiometric.

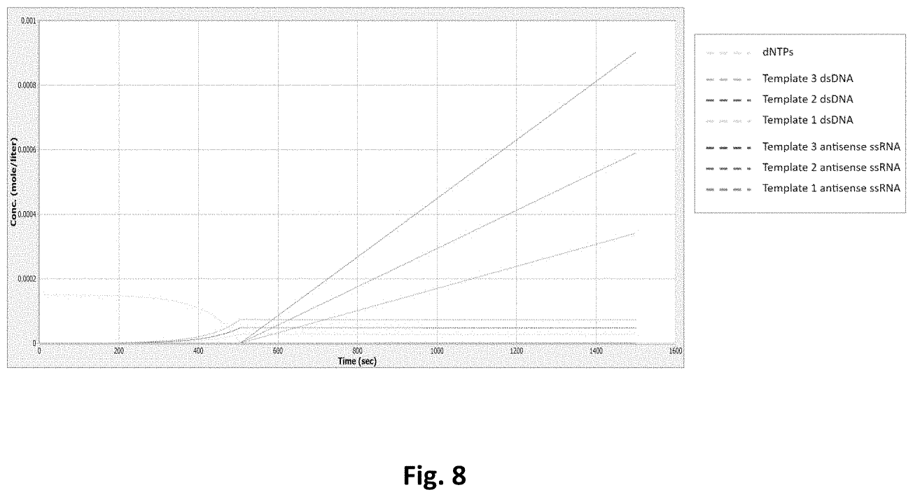

As may be seen in FIG. 8, a reaction simulation in kinetiscope produces a graphical representation of component concentrations (e.g., dNTPs, dsDNA, and ssRNA). In this simulation, the starting NASBA components were adjusted so that dNTPs would be rate limiting. Three different input genes were added to the reaction at initial ratios of 1×(template 1), 2×(template 2) and 3×(template 3). From this graph one can see that when dNTPs (light blue) are depleted, exponential production of double stranded cDNA (grey, purple, light green) stops. At the same time linear amplification of antisense RNA (dark red, dark green, pink) begins. The ratios of the final concentrations of the amplified antisense RNAs are the same (1×, 2×, 3×) to the initial ratios of the input RNAs.

Conclusion: A simulation of the system described herein with relative rate constants and concentrations comparable to the experimental values shows a first regime of template amplification. Once the dNTP pool has been exhausted, this gives way to a second regime where oligonucleotides are amplified linearly in time with their initial relative abundances preserved (i.e., ratiometrically).

### DOCTRINE OF EQUIVALENTS

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

1. A method for ratiometric amplification of specific nucleic acids, comprising:
obtaining or having obtained a sample, wherein the sample contains nucleic acids; and
performing an amplification reaction on the sample to amplify one or more target sequences in the sample by:
combining the sample with reaction components, wherein the reaction components comprise at least one target specific primer, nucleotides, and at least one polymerase, and
incubating the sample and the reaction components.

2. The method of claim 1, wherein the incubating step is performed isothermally.

3. The method of claim 1, wherein the incubating step is performed with an initial heating step, then incubated isothermally.

4. The method of claim 1, further comprising analyzing an output of the amplification reaction.

5. The method of claim 4, wherein the analyzing step is performed using a fluorescence assay.

6. The method of claim 4, further comprising analyzing an output of the amplification reaction to identify one or more target sequences.

7. The method of claim 4, wherein the analyzing step is performed using electrophoresis.

8. The method of claim 7, wherein the electrophoresis is selected from the group consisting of gel electrophoresis and capillary electrophoresis.

9. The method of claim 7, wherein the electrophoresis is capable of quantitatively analyzing the output components.

10. The method of claim 1, wherein the amplification reaction is selected from the group consisting of: nucleic acid sequence based amplification (NASBA), Loop-mediated isothermal amplification, Recombinase Polymerase Amplification, Rolling Circle Amplification, and Nicking Enzyme Amplification Reaction.

11. The method of claim 4, wherein the analyzing step includes diagnosing an individual for a disease or disease state.

12. The method of claim 11, wherein the disease or disease state is selected from the group consisting of HIV, tuberculosis, and sepsis.

13. The method of claim 1, wherein the reaction components comprise dNTPs, NTPs, a buffer, at least one primer, and a transcriptase.

14. The method of claim 13, wherein the components further include a reverse transcriptase.

15. The method of claim 13, wherein the transcriptase is an RNA polymerase.

16. The method of claim 13, wherein the components further include a nuclease.

17. The method of claim 16, wherein the nuclease is RNase H.

18. The method of claim 13, wherein the dNTPs are a limiting reagent in the reaction to maintain ratiometric amplification of the template nucleic acids in the sample.

19. The method of claim 18, wherein the dNTP concentration is no greater than ⅕× concentration for a standard amplification reaction.

20. The method of claim 1, wherein the biological sample is selected from the group consisting of saliva, urine, and blood.
